(19) United States
(12) Patent Application Publication
(10) Pub. No.: US 2023/0408332 A1
(43) Pub. Date: Dec. 21, 2023

(54) OPTICAL FIBER SURFACE ELECTRIC FIELD SCREENING APPARATUS

(71) Applicant: LIAOCHENG UNIVERSITY, Liaocheng (CN)

(72) Inventors: Nan-Kuang CHEN, Liaocheng (CN); Yucheng LI, Liaocheng (CN); Haimiao ZHOU, Liaocheng (CN); Zhengkai YAO, Liaocheng (CN); Haili HAN, Liaocheng (CN); Yicun YAO, Liaocheng (CN); Shijie REN, Liaocheng (CN); Hongzhu JI, Liaocheng (CN)

(21) Appl. No.: 17/754,490

(22) PCT Filed: Jun. 19, 2020
(86) PCT No.: PCT/CN2020/097178

§ 371 Date: Apr. 4, 2022

(30) Foreign Application Priority Data
Nov. 11, 2019 (CN) 201911097015.3

(51) Int. Cl. G01J 3/02

(52) U.S. Cl. CPC G01J 3/0218; G01J 3/0208; C12M 47/02

(57) ABSTRACT

An optical fiber surface electric field screening apparatus, comprising an extractor, the extractor being provided with an optical fiber, the optical fiber being prepared so as to form in the middle thereof a thinned part having a diameter of 15 μm or less, causing a surface of the thinned part to generate a negative electric charge, capable of being used to attract a cell having a surface carrying a positive electric charge, e.g., attracting a cardiac cell, an endothelial cell or a bacterium to surround the thinned part, achieving the effect of screening and extracting cells.

### TECHNICAL FIELD

The present disclosure relates to innovative application of optical fiber electrification, and in particular relates to an optical fiber surface electric field screening apparatus.

### BACKGROUND ART

As normal cells and diseased cells cannot be screened in the prior art, the general practice of killing diseased cells is to kill normal cells and diseased cells together, however, such practice may damage the immune system of the human, leading to weakened immunity.

Moreover, when the diseased cells are studied, normal cells and diseased cells can be screened only if the diseased cells can be observed from the appearance, and the above conditions make it difficult to kill the diseased cells or study the diseased cells.

### SUMMARY

To solve the problem that normal cells and diseased cells cannot be screened by an existing method, the optical fiber electrification technology is used for innovative application, and an optical fiber surface electric field screening apparatus is provided by the present disclosure. The optical fiber surface electric field screening apparatus comprises an extractor, the extractor is provided with an optical fiber, the optical fiber is prepared to form, in the middle thereof, a thinned part having a diameter of 15 μm or less, and a negative electric charge is generated on a surface of the thinned part.

Preferably, the periphery of the thinned part is covered with a layer of organic material.

Further, both ends of the optical fiber are connected to an optical source and a spectrograph, respectively.

Furthermore, the light source is a super-luminescent diode.

Further, the optical fiber surface electric field screening apparatus comprises an electric field generating apparatus cooperating with the extractor, the electric field generating apparatus is provided with a high-voltage power supply, a positive electrode of the high-voltage power supply is electrically connected to a conductive structure to power the conductive structure to generate an electric field, and then the electric field is applied to the thinned part of the extractor.

Preferably, the optical fiber is a single-mode optical fiber or a seven-core optical fiber.

During use, the tapered thinned part having the surface with the negative electric charge formed thereon stretches to a position where a diseased cell needs to be extracted, then the diseased cell having a surface with a positive electric charge is extracted by the thinned part, thus the diseased cell can be extracted from a tissue.

The present disclosure has the beneficial effects that an optical fiber serving as an extractor is machined to form a thinned part having a diameter of 6 μm, then a negative electric charge can be generated on a surface of the thinned part; the thinned part with the negative electric field of the extractor can be used for the operations of attracting and extracting the diseased cell, and is especially suitable for separating the diseased cell having the surface with the positive electric charge from a normal cell with a negative electric charge, e.g., extracting the diseased cell and a bacterium each having a surface with the positive electric charge, or extracting the bacterium and diseased cell each having the surface with the positive electric charge from a sample during an experiment for studying.

An electric field generating apparatus further included by the present disclosure can further apply an electric field to the thinned part of the extractor, causing the surface of the thinned part to generate more negative electric charges, thus improving the efficacy of the extractor to extract the diseased cells and bacteria having surfaces with the positive electric charges.

The thinned part with the negative electric charge of the extractor can attract any substances with the positive electric charge, and can also attract an inorganic matter with the positive electric charge such as soot particles in addition to attracting and extracting the cells with the positive electric charge; the particles adsorbed by the thinned part may hinder an effect of the optical fiber to transmit an optical fiber, thus, in a way of connecting the light source and the spectrograph to both ends of the optical fiber to send the optical signal at one end while receiving the optical signal at other end, the number of the particles adsorbed by the thinned part of the extractor can be confirmed by reading a numerical value of the spectrograph.

### BRIEF DESCRIPTION OF THE DRAWINGS

FIG. 1 is a diagram of an extractor of a preferred embodiment of the present disclosure;

FIG. 2 is a diagram of an apparatus of a preferred embodiment of the present disclosure; and

FIG. 3 is a graph of wavelength optical power loss spectra for attracting particles according to a preferred embodiment of the present disclosure.

### IN THE DRAWINGS

10—extractor; 11—optical fiber; 12—thinned part; 13—organic material; 20—electric field generating device; 21—high—voltage power supply; 22—conductive structure; 30—light source; 40—spectrograph; A—electric field; B—negative electric charge.

### DETAILED DESCRIPTION OF THE EMBODIMENTS

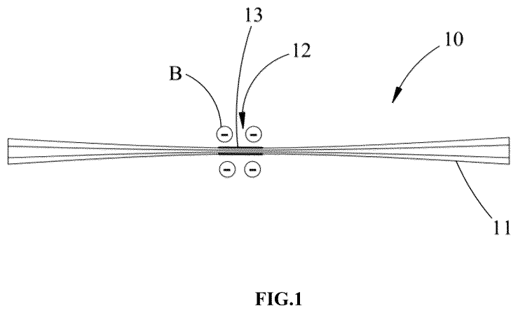

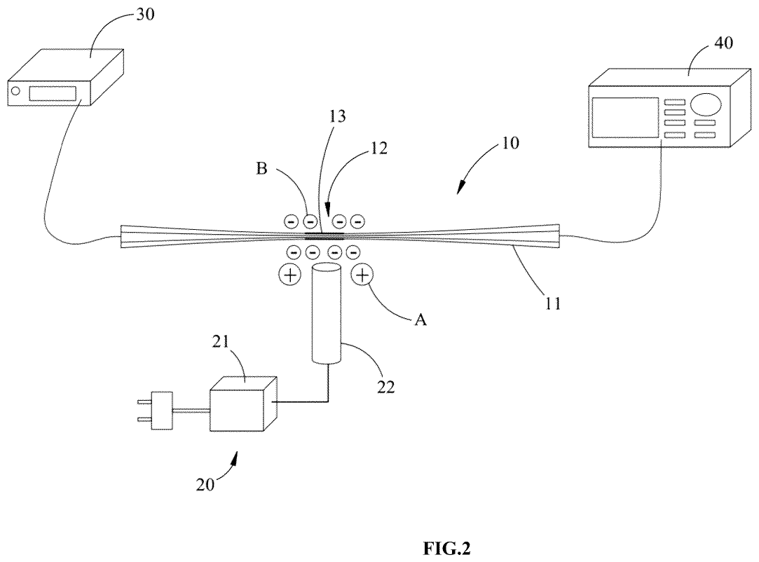

Please referring to the drawings of the preferred embodiments shown in FIG. 1 and FIG. 2, an optical fiber surface electric field screening apparatus provided by the present disclosure comprises an extractor 10, an electric field generating apparatus 20 cooperating with the extractor 10 for generating an electric field, a light source 30, and a spectrograph 40 (OSA), wherein:
the extractor 10 is provided with an optical fiber 11, which may be a single-mode optical fiber or a seven-core optical fiber; and the optical fiber 11 have two opposite ends. In the preferred embodiment, the single-mode optical fiber is adopted as the optical fiber 11, a thinned part 12 is formed in the middle of the optical fiber 11 by tapering and machining by an optical fiber fused biconical taper machine; in a dry and oxygen-free environment, the optical fiber 11 is tapered and machined to form a thinned part 12 having a diameter of 15 μm and less, causing a surface of the thinned part 12 to generate a negative electric charge B; in an indoor environment, the optical fiber 11 is tapered and machined to form a thinned part 12 having a diameter of 6 μm and less, causing a surface of the thinned part 12 to generate a negative electric charge 6. In the preferred embodiment, the extractor 10 is applied to a general indoor environment, the diameter of the thinned part 12 formed by machining the optical fiber 11 is 3.97 μm; moreover, in order to adsorb cells or bacteria better, the surrounding of the thinned part 12 is covered with a layer of organic material 13 with cell affinity in the preferred embodiment, e.g., table vinegar, 50% concentrated sulfuric acid or potassium hydroxide.

The electric field generating apparatus 20 is a selectively arranged apparatus and is used to reinforce the negative electric charge B on the surface of the thinned part 12. The electric field generating apparatus 20 is provided with a high-voltage power supply 21 to which alternating current is supplied; a positive electrode of the high-voltage power supply 21 is electrically connected to a conductive structure 22, in the preferred embodiment, the conductive structure 22 is a copper wire, and the high-voltage power supply 21 supplies direct current of 5 kV to the conductive structure 22 to make the conductive structure generate an electric field A, then the electric field A is applied to the thinned part 12 of the extractor 10 to reinforce the negative electric charge B on the surface of the thinned part 12.

The light source 30 and the spectrograph 40 are also selectively arranged apparatuses; during use, the light source 30 and the spectrograph 40 are connected to two opposite ends of the optical fiber 11, respectively; the light source 30 is a super-luminescent diode (SLD), the light source 30 outputs an optical signal to the optical fiber 11 of the extractor 10, and the optical signal is received by the spectrograph 40 after passing through the thinned part 12; the number of particles adsorbed to the thinned part 12 may affect the transmission of the optical signal by the optical fiber 11, and the more particles adsorbed in the thinned part 12, the greater the optical power loss of the transmission of the optical signal by the optical fiber 11.

When the preferred embodiment is used to adsorb a diseased cell or a bacterium having a surface with positive electric charge, if only the extractor 10 is used, the thinned part 12 of the extractor is close to the cell, the thinned part 12 having the surface with the negative electric charge is used to attract and extract the diseased cell or bacterium having the surface with the positive electric charge, the diseased cell or bacterium is separated from the normal cells and adsorbed to the organic material 12 surrounding the thinned part 12; whereby, the diseased cell or bacterium can be extracted from a tissue or a sample without damaging the normal cells.

When the extractor 10 is cooperated with the electric field generator 20 for use, the electric field A generated by the electric field generator 20 is applied to a position where diseased cells or bacteria need to be extracted, i.e., the range that the thinned parts 12 of the extractor 10 extract the cells; more negative electric charges may be generated to surround the thinned part 12 in the electric field A, then the thinned part 12 having the surface with the negative electric charges B is used to attract and extract the diseased cells or bacteria having the surfaces with the positive electric charges, and these diseased cells or bacteria can be extracted from the tissue or a sample without damaging the normal cells.

When the preferred embodiment is used to adsorb particles, the extractor 10 can be used to adsorb particles alone, or the thinned part 12 can be used to adsorb particles by placing the thinned part 12 of the extractor 10 within the range of action of the electric field A, and then the thinned part 12 can be used to adsorb particles directly without providing a layer of organic material 13 surrounding the thinned part 12.

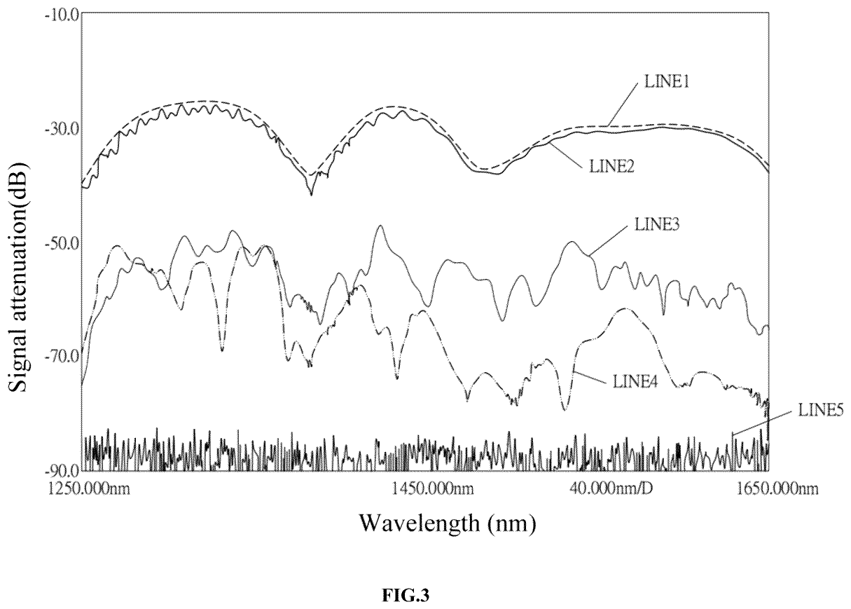

The light source 30 outputs an optical signal to the optical fiber 11 of the extractor 10, the optical signal is received by the spectrograph 40 after passing through the thinned part 12, and then a wavelength loss spectral graph as shown in FIG. 3 is output, wherein LINE′ is waveform of the optical fiber 11 which is not tapered, LINE2 is the waveform of the tapered optical fiber 11, LINE3, LINE4 and LINE5 are waveforms after the thinned part 12 adsorb the soot for 20 seconds, 30 seconds, and 40 seconds. As shown in FIG. 2, when the light source 30 outputs the optical signal to the optical fiber 11 of the extractor 10 and the optical signal is received by the spectrograph 40 after passing through the thinned part 12, the more particles adsorbed by the thinned part 12, the greater the optical power loss of the transmission of the optical fiber 11, the number of particles absorbed by the thinned part 12 can be judged by reading the spectrogram displayed by the spectrograph 40.

In addition to tapering and machining the optical fiber 11 to form a thinned part 13 in the preferred embodiment, the optical fiber 11 can be machined to form the thinned part 13 by means of chemical etching or mechanical processing and grinding. During use, it is also possible to provide a negative electric charge surrounding the thinned part 12 for extracting the diseased cells or bacteria having the surface with positive electric charge.

The foregoing is merely preferred embodiments of the present disclosure and is not intended to limit the claimed scope of the present disclosure, and other equivalent changes or modifications made without departing from the spirit of the present disclosure should be included within the claimed scope of the present disclosure.

1.-7. (canceled)

8. An optical fiber surface electric field screening apparatus, comprising an extractor, wherein the extractor is provided with an optical fiber, the optical fiber is prepared to form, in the middle thereof, a thinned part having a diameter of 15 μm or less, and a negative electric charge is generated on a surface of the thinned part.

9. The optical fiber surface electric field screening apparatus according to claim 8, wherein the thinned part is surrounded with a layer of organic material.

10. The optical fiber surface electric field screening apparatus according to claim 8, wherein both ends of the optical fiber are connected to a light source and a spectrograph, respectively.

11. The optical fiber surface electric field screening apparatus according to claim 10, wherein the light source is a super-luminescent diode.

12. The optical fiber surface electric field screening apparatus according to claim 8, wherein the apparatus further comprises an electric field generating apparatus cooperating with the extractor, the electric field generating apparatus is provided with a high-voltage power supply, a positive electrode of the high-voltage power supply is electrically connected to a conductive structure to power the conductive structure to generate an electric field, and then the electric field is applied to the thinned part of the extractor.

13. The optical fiber surface electric field screening apparatus according to claim 9, wherein the apparatus further comprises an electric field generating apparatus cooperating with the extractor, the electric field generating apparatus is provided with a high-voltage power supply, a positive electrode of the high-voltage power supply is electrically connected to a conductive structure to power the conductive structure to generate an electric field, and then the electric field is applied to the thinned part of the extractor.

14. The optical fiber surface electric field screening apparatus according to claim 10, wherein the apparatus further comprises an electric field generating apparatus cooperating with the extractor, the electric field generating apparatus is provided with a high-voltage power supply, a positive electrode of the high-voltage power supply is electrically connected to a conductive structure to power the conductive structure to generate an electric field, and then the electric field is applied to the thinned part of the extractor.

15. The optical fiber surface electric field screening apparatus according to claim 8, wherein the optical fiber is a single-mode optical fiber or a seven-core optical fiber.

16. The optical fiber surface electric field screening apparatus according to claim 9, wherein the optical fiber is a single-mode optical fiber or a seven-core optical fiber.

17. The optical fiber surface electric field screening apparatus according to claim 10, wherein the optical fiber is a single-mode optical fiber or a seven-core optical fiber.

18. The optical fiber surface electric field screening apparatus according to claim 10, wherein the optical fiber is a single-mode optical fiber or a seven-core optical fiber.

19. The optical fiber surface electric field screening apparatus according to claim 12, wherein the optical fiber is a single-mode optical fiber or a seven-core optical fiber.

20. The optical fiber surface electric field screening apparatus according to claim 13, wherein the optical fiber is a single-mode optical fiber or a seven-core optical fiber.

21. The optical fiber surface electric field screening apparatus according to claim 14, wherein the optical fiber is a single-mode optical fiber or a seven-core optical fiber.
